(19) United States
(12) Patent Application Publication
(10) Pub. No.: US 2021/0386622 A1
(43) Pub. Date: Dec. 16, 2021

(54) FLUID COUPLINGS

(71) Applicant: Colder Products Company, Roseville, MN (US)

(72) Inventors: Kevin John Cornelius, Wayzata, MN (US); Elizabeth J. Langer, Minneapolis, MN (US); Krista A. Vangsgard, Oakdale, MN (US); Erik Anthony Menne Weigel, Minneapolis, MN (US)

(21) Appl. No.: 17/331,202

(22) Filed: May 26, 2021

Related U.S. Application Data
(60) Provisional application No. 63/038,321, filed on Jun. 12, 2020.

(51) Int. Cl. A61J 1/20; A61J 1/14; F16L 37/367; F16L 37/127

(52) U.S. Cl. CPC A61J 1/2048; F16L 37/127; F16L 37/367; A61J 1/1443

(57) ABSTRACT

Some fluid coupling devices described herein are configured for use in fluid systems for purposes of providing a sterile connection for drug delivery. In some embodiments, the fluid coupling devices can be implemented as multi-use, sterile fluid coupling devices that are configured to reduce the likelihood of fluid spillage when being disconnected.

### CROSS-REFERENCE TO RELATED APPLICATIONS

This application claims the benefit of U.S. Provisional Application Ser. No. 63/038,321, filed Jun. 12, 2020. The disclosure of the prior application is considered part of (and is incorporated by reference in) the disclosure of this application.

### BACKGROUND

### 1. Technical Field

This document relates to fluid coupling devices for fluid systems and methods of using the fluid coupling devices. For example, some embodiments described in this document relate to genderless fluid couplings that can be used to provide a sterile connection for drug delivery.

### 2. Background Information

Fluid systems commonly include components such as tubing, pumps, reservoirs, fittings, couplings, heat exchangers, sensors, filters, valves, seals, and the like. Such components can be connected together in a network to define one or more fluid flow paths. Some fluid systems are open systems, meaning that the fluid flows through the network once and then exits the network. Other fluid systems are closed systems, meaning that the fluid recirculates within the network of components. Fluids may be moved through fluid systems using fluid pressure differentials. For example, in some cases, a pump or a vacuum source is used to create a pressure differential that causes the fluid to flow within the fluid system. In another example, gravity is used to cause the fluid to flow within the fluid system. In other examples, a combination of such techniques is used to cause the fluid to flow within the fluid system.

Some fluid couplings can be used in a medical context, such as for connecting a source of a therapeutic agent (or “drug”) to an intravenous drug delivery system. In some cases, the steps for intravenously delivering a drug to a patient can include: (i) connecting a syringe to a bottle or vial containing a drug, (ii) transferring an amount of the drug from the bottle into the syringe, (iii) disconnecting the syringe from the bottle, (iv) connecting the syringe to an intravenous (IV) bag and/or tubing, and then (v) delivering the drug via the IV tubing to the patient.

### SUMMARY

This document describes fluid coupling devices for fluid systems and methods. For example, in some embodiments this document describes genderless fluid couplings that can be used to provide a sterile connection for drug delivery.

In some embodiments, the fluid coupling devices can be implemented as multi-use, sterile fluid coupling devices that are configured to reduce the likelihood of fluid spillage when being disconnected. The fluid coupling devices described herein also minimize dead space so that a minimal amount of fluid is retained in the coupling devices after use.

Additionally, in some such multi-use embodiments or in other embodiments, the fluid coupling devices can be configured as “sterile” or “aseptic” coupling devices in that, after the two portions of the coupling device are disconnected from each other, the fluid paths of both portions are mechanically blocked so as to inhibit biological contamination migrating into the flow paths. Such an “aseptic” coupling will also serve to limit the exposure of the fluid to the surrounding environment.

Further, in such multi-use embodiments, or other embodiments, the fluid coupling devices can be configured as no-spill coupling devices because, as the two portions of the coupling device are being disconnected from each other, one or more mechanical components will reduce the likelihood of fluid discharge out of the fluid system (for example, by blocking as such discharge paths).

In one aspect, this disclosure is directed to a fluid coupling device that includes: (i) a housing defining an internal space and a lumen configured for fluid flow, the lumen defining a central axis; (ii) a seal member affixed to the housing and including an upper surface exposed to the internal space, the seal member defining a through-hole in alignment with the lumen; and (iii) a shuttle disposed within the internal space and slidable relative to the housing along a path that intersects the central axis. The shuttle is slidable between: (i) a first position in which the shuttle is abutted against the upper surface of the seal member to cover the through-hole and prevent fluid flow through the lumen, and (ii) a second position in which the shuttle is spaced apart from and out of contact with the seal member.

Such a fluid coupling device may optionally include one or more of the following features. The fluid coupling device may also include a spring disposed between the housing and the shuttle. The spring may bias the shuttle toward the first position. The fluid coupling may also include a latch member attached to the housing. The latch member may detain the shuttle member in the first position as the spring pushes the shuttle member into contact against the latch member. The latch member may be manually pivotable relative to the shuttle member. The path that the shuttle slides along relative to the housing may be orthogonal to the central axis. The seal member may include a circular rib that is concentric with the through-hole. In some embodiments, the seal member includes two circular ribs that are each concentric with the through-hole. In some embodiments, while the shuttle is in the second position, the through-hole is open to the internal space. The cross-sectional shape of the internal space taken parallel to the central axis may be semicircular. The cross-sectional shape of the shuttle taken parallel to the central axis may be semicircular. The housing may have a semicircular front face with a size that matches the cross-sectional shape of the shuttle.

In another aspect, this disclosure is directed to a fluid coupling system that includes a first fluid coupling device and a second fluid coupling device that is identical to the first fluid coupling device. The first fluid coupling device may include a first housing defining a first internal space and a first lumen configured for fluid flow, the first lumen defining a first central axis; a first seal member affixed to the first housing and including a first upper surface exposed to the first internal space, the first seal member defining a first through-hole in alignment with the first lumen; and a first shuttle disposed within the first internal space and slidable relative to the first housing along a first path that intersects the first central axis, the first shuttle being slidable between: (i) a first position in which the first shuttle is abutted against the first upper surface of the first seal member to cover the first through-hole and prevent fluid flow through the first lumen, and (ii) a second position in which the first shuttle is spaced apart from and out of contact with the first seal member.

Such a fluid coupling system may optionally include one or more of the following features. In some embodiments, the first and second fluid couplings are slidable relative to each other into a coupled arrangement that defines an open fluid flow path therethrough. The first housing and the second housing may each have a front face. A shape of the front face may match a shape of an end of the first shuttle and an end of the second shuttle. The first and second fluid coupling devices may be coupleable by: (i) aligning the first and second fluid coupling devices such that the front face of the first housing is in contact with the second shuttle and the front face of the second housing is in contact with the first shuttle, and (ii) pushing the first and second fluid coupling devices toward each other to slide the first and second shuttles to their second positions. When the first and second shuttles are in their second positions the first and second through-holes may be in alignment with each other. The first fluid coupling device may include a first latch member, and the second fluid coupling device may include a second latch member. When the first and second shuttles are in their second positions: (i) the first latch member may be releasably engaged with the second housing and (ii) the second latch member may be releasably engaged with the first housing. The first latch member may be manually pivotable relative to the first housing, and the second latch member may be manually pivotable relative to the second housing. When the first and second fluid coupling devices are uncoupled from each other, a first spring of the first fluid coupling device may force the first shuttle into engagement against the first latch member and the first latch member may detain the first shuttle in the first position against the forces of the first spring.

Particular embodiments of the subject matter described in this document can be implemented to realize one or more of the following advantages. First, in some embodiments the fluid coupling devices provided herein are configured to facilitate a greater number of sterile connections as compared to conventional connection means (e.g., a needle piercing a silicon membrane or septum).

Second, in some embodiments, the fluid coupling devices described herein may advantageously reduce the amount of dead volume in the fluid coupling system.

Third, some embodiments of the fluid coupling devices provided herein are genderless couplings, meaning that a pair of two single couplings that are identical are conveniently used to make the fluid connection, rather than requiring specific male and female couplings.

Fourth, some embodiments of the fluid coupling devices provide an improved aseptic connection and disconnection capability that may optionally reduce or eliminate the need for sterile rooms or sterile benchtop environments in some cases. As such, these embodiments of the aseptic fluid coupling devices described herein may facilitate efficient and cost-effective operations or uses that would otherwise be high-cost or even cost prohibitive in some traditional settings that required the connection and/or disconnection of particular fluid couplings in a sterile room or within a sterile flow-hood to prevent biological contamination.

Fifth, some embodiments of the fluid coupling devices provided herein are advantageously designed with a robust latching system. That is, when the two halves of the coupling are operably connected with each other, they are also mechanically latched or locked together. In some embodiments, to release the lock, two levers must be simultaneously depressed. This redundant requirement (e.g., simultaneous actuation of two levers or other actuators) for unlocking the coupling halves may reduce the likelihood of unintentional disconnections.

Sixth, in some embodiments the fluid couplings described herein are configured to reduce the likelihood of fluid spillage when being disconnected. Further, the fluid couplings described herein are designed to prevent the inclusion of air into the fluid, as can often result during the process of joining male and female couplings together.

Seventh, some embodiments do not have springs in the flow path.

Eighth, some embodiments have a smooth, unobstructed flow path.

In the context of this disclosure, the term “fluid” includes gases, liquids, and powders.

Unless otherwise defined, all technical and scientific terms used herein have the same meaning as commonly understood by one of ordinary skill in the art to which this disclosure pertains. In addition, the materials, methods, and examples of the embodiments described herein are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of an example single fluid coupling device, in accordance with some embodiments provided herein.

FIG. 2 is another perspective view of the fluid coupling device of FIG. 1.

FIG. 3 is a first side view of the fluid coupling device of FIG. 1.

FIG. 4 is a top view of the fluid coupling device of FIG. 1.

FIG. 5 is an end view of the fluid coupling device of FIG. 1.

FIG. 6 is a longitudinal cross-sectional view showing two of the fluid coupling devices of FIG. 1 in alignment in preparation for being coupled together.

FIG. 7 is a longitudinal cross-sectional view showing two of the fluid coupling devices of FIG. 1 coupled together in an operative configuration.

FIG. 8 is a perspective view showing two of the fluid coupling devices of FIG. 1 coupled together in an operative configuration.

FIG. 9 is a side view showing two of the fluid coupling devices of FIG. 1 coupled together in an operative configuration.

FIG. 10 is a top view showing two of the fluid coupling devices of FIG. 1 coupled together in an operative configuration.

FIG. 11 is an end view showing two of the fluid coupling devices of FIG. 1 coupled together in an operative configuration.

FIG. 12 is a perspective view of an example seal member of the fluid coupling device of FIG. 1.

FIG. 13 is a perspective view of an example housing and seal member of the fluid coupling device of FIG. 1.

Like reference numbers represent corresponding parts throughout.

### DETAILED DESCRIPTION OF ILLUSTRATIVE EMBODIMENTS

This document describes fluid coupling devices for fluid systems and methods. For example, in some embodiments this document describes genderless fluid couplings that can be used to provide a sterile connection for drug delivery. In some embodiments, the fluid coupling devices can be implemented as multi-use, sterile fluid coupling devices that are configured to reduce the likelihood of fluid spillage when being disconnected.

The couplings and coupling systems described herein solve, for example, some issues related to repeatably providing a sterile connection for drug delivery. The couplings and coupling systems described herein achieve this by use of a wiping seal and a minimum number of components. The coupling device is also a genderless connector (meaning there are no traditional male and female halves). These attributes (e.g., the minimum number of components and the genderless arrangement) combine to provide a solution that can be mass manufactured cost-effectively.

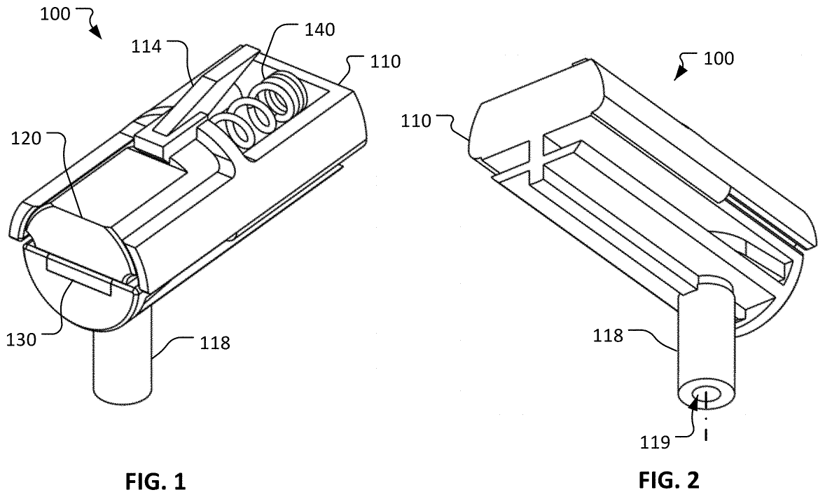

Referring to FIGS. 1-5, an example fluid coupling 100 includes a housing 110, a shuttle 120, a seal member 130, and a spring 140. As described further below, a pair of the fluid couplings 100 can be coupled together to form an open fluid flow path therethrough. Because two of the fluid couplings 100 (which can be identical to each other) can be coupled together, it can be said that the fluid coupling 100 is a “genderless” fluid coupling.

The materials from which one or more of the components of the fluid coupling 100 are made of include thermoplastics. In particular embodiments, the materials from which the components of the fluid coupling 100 are made of are thermoplastics, such as, but not limited to, acetal, polycarbonate, polysulfone, polyether ether ketone, polysulphide, polyester, polyvinylidene fluoride (PVDF), polyethylene, polyphenylsulfone (PPSU; e.g., Radel®), polyetherimide (PEI; e.g., Ultem®), polypropylene, polyphenylene, polyaryletherketone, and the like, and combinations thereof.

In some embodiments, the materials from which one or more of the components of the fluid coupling 100 are made of include metals such as, but not limited to stainless steel, brass, aluminum, plated steel, and the like. In particular embodiments, the fluid coupling 100 is metallic-free.

In some embodiments, the spring 140 is made of a metallic material (e.g., spring steel, stainless steel such as 316L, and the like). In some embodiments, the spring 140 can be made of a polymeric or elastomeric material.

In certain embodiments, the seal member 130 can be made of materials such as, but not limited to, silicone, fluoroelastomers (FKM), ethylene propylene diene monomer (EPDM), thermoplastic elastomers (TPE), buna, buna-N, thermoplastic vulcanizates (TPV), and the like.

The shuttle 120 is slidably coupled in relation to the housing 110. That is, the shuttle 120 can slide within a space defined by the housing 110.

The spring 140 is disposed between the housing 110 and the shuttle 120. The spring 140 biases the shuttle 120 to its first end of travel position (as shown) and resists (but allows) the shuttle 120 to be slid in relation to the housing 110 toward its opposite, second end of travel position. As the shuttle 120 is slid in relation to the housing 110 (away from its depicted end of travel position) the spring 140 become further compressed.

The housing 110 includes a fluid connection or termination 118 that can be configured in any desired manner (e.g., as a luer fitting, a barbed connection, a threaded connection, as any type of adapter, a sanitary fitting, etc.). The termination 118 defines a lumen 119. The lumen 119 provides the fluid flow path of the fluid coupling 100 when two fluid couplings 100 are fully coupled together (as described further below). The lumen 119 is blocked and fluidly sealed-off by the shuttle 120 in the depicted configuration.

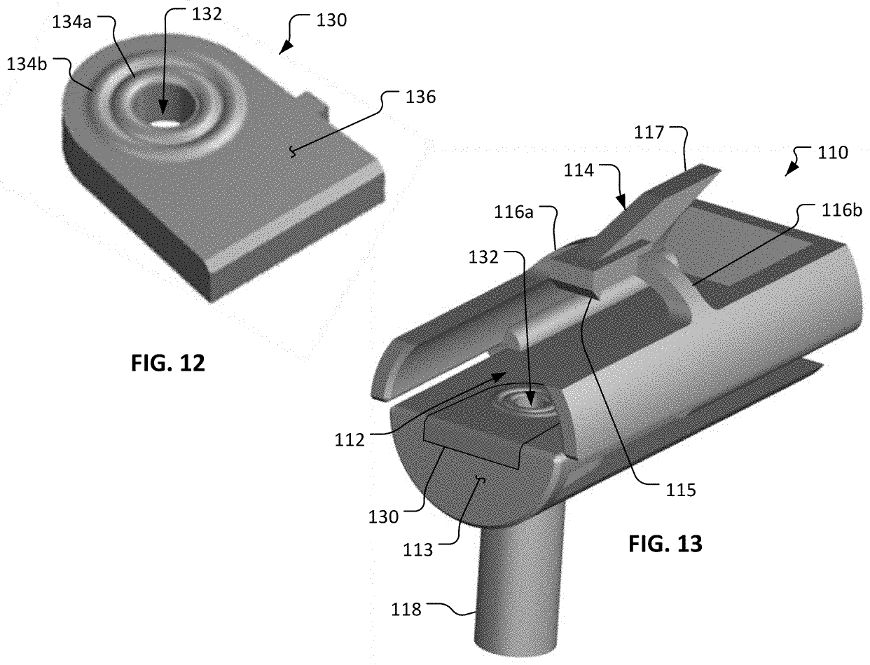

Referring also to FIGS. 12 and 13, the seal member 130 is affixed to the housing 110. In some embodiments, the seal member 130 is overmolded onto the housing 110. In some embodiments, the seal member 130 is attached to the housing 110 in another manner, such as by using an adhesive, ultrasonic welding, press-fitting, and the like.

The seal member 130 defines a through-hole 132. The through-hole 132 is aligned with the lumen 119. Accordingly, the lumen 119 and the through-hole 132 of the seal member 130 provide the fluid flow path of the fluid coupling 100 when two fluid couplings 100 are fully coupled together (as described further below).

When the shuttle 120 is in its first end of travel position (e.g., as depicted in FIGS. 1-6), the shuttle 120 engages against the seal member 130 to block fluid from flowing through the fluid coupling 100.

As shown in FIG. 12, the seal member 130 can include one or more circular ribs that are concentric with the through-hole 132. In the depicted embodiment, the seal member 130 includes two circular ribs 134a and 134b that are concentric with the through-hole 132. These circular ribs 134a and 134b project above the primary upper surface 136 of the seal member 130. Accordingly, the circular ribs 134a and 134b act as gaskets that facilitate a wiping, fluid-tight seal with the shuttle 120 when the shuttle 120 is in its first end of travel position.

FIG. 13 shows that the housing 110 defines an internal space 112 that the shuttle 120 (not shown in FIG. 13) is slidably disposed within. The shuttle 120 slides along a linear path within the internal space 112 between its first end position and its second end position. The linear path intersects the central axis of the lumen 119 (FIG. 2). The linear path that the shuttle 120 slides along is nonparallel with the central axis of the lumen 119. In the depicted embodiment, the linear path that the shuttle 120 slides along is orthogonal to the central axis of the lumen 119. The upper surface 136 of the seal member 130 is exposed to the internal space 112.

In the depicted embodiment, the cross-sectional shape of the internal space 112 is semicircular (and the shuttle 120 has a slightly smaller semicircular outer profile). In addition, the cross-sectional shape of a front face 113 of the housing is also semicircular and the same size as the shuttle 120. Alternatively, other cross-sectional shapes can be used such as, but not limited to, rectangular, triangular, polygonal, and so on.

Still referring in particular to FIG. 13, the housing 110 also includes a latch member 114 and latch support members 116a and 116b. The latch support members 116a and 116b are each attached to the latch member 114 (attached on opposite sides of the latch member 114).

The latch member 114 includes a first end that includes at least one projection 115 and an opposite, second end that includes a depressible arm 117. The latch support members 116a and 116b are each attached to the latch member 114 between the first and second ends of the latch member 114.

When the depressible arm 117 is manually depressed by a user of the fluid coupling 100, the latch member 114 will pivot. As the latch member 114 pivots, the first end that includes the at least one projection 115 will pivot in an upward direction away from the seal member 130 (in a direction opposite of the depressible arm 117 as it is depressed).

In the depicted embodiment, when the depressible arm 117 is manually depressed the latch support members 116a and 116b will twist to allow the latch member 114 to pivot. Then, when the force from the manual depression of the depressible arm 117 is removed, the latch support members 116a and 116b will rebound (naturally untwist) to return the latch member 114 to the depicted arrangement. In some embodiments, the housing 110 (including the latch member 114 and latch support members 116a and 116b) is a monolithic, unitary component (e.g., injection molded). Accordingly, the latch support members 116a and 116b can be said to provide a living hinge (or living torsion springs) type of connection for the latch member 114 to the rest of the housing 110. In some embodiments, the housing 110 can be constructed from separate but coupled components rather than being monolithic.

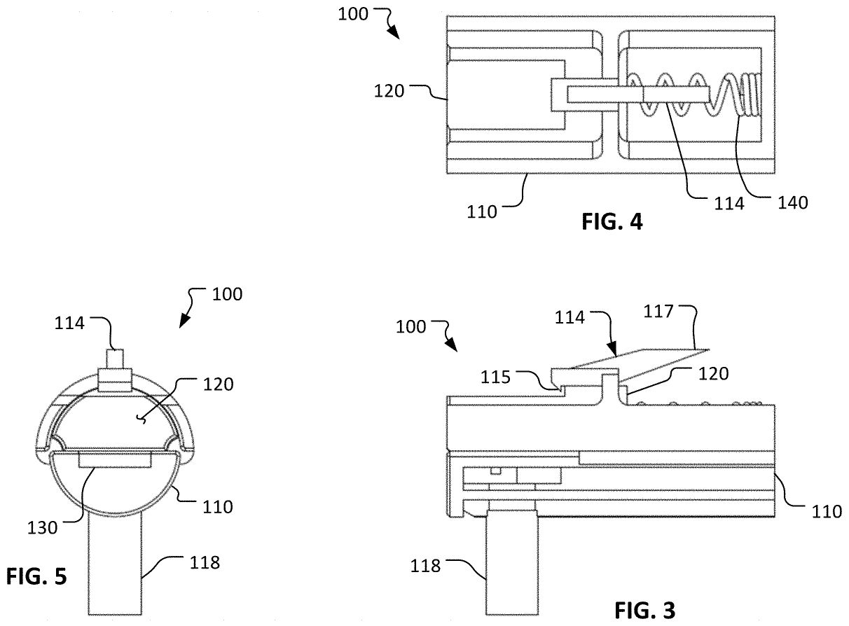

As visible in FIG. 3, for example, the at least one projection 115 of the latch member 114 engages against the shuttle 120 while the shuttle 120 is in its first end of travel position. That is, the at least one projection 115 of the latch member 114 limits the travel of the shuttle 120 (or blocks the shuttle 120 from being pushed farther by the spring 140) such that the shuttle 120 does not travel out of engagement with the housing 110.

Now referring to FIG. 6, cross-sectional views of a first coupling 100a and a second coupling 100b (each of which are the same as the fluid coupling 100 as described herein) are shown in alignment in preparation for becoming coupled together. It can be seen that the couplings 100a and 100b are arranged so that the shuttle 120a of the first coupling 100a is upward and the shuttle 120b of the second coupling 100b is downward.

In this cross-sectional view, it can be seen that the shuttle 120a is engaged against the seal member 130a, and the shuttle 120b is engaged against the seal member 130b. Accordingly, the lumens 119a and 119b are blocked and fluidly sealed at the interface between the shuttles 120a-b and seal members 130a-b. The shuttles 120a-b and seal members 130a-b act as valves.

In this view, it can also be seen that the housing 110a of the first coupling 100a defines a groove 111a, and the housing 110b of the second coupling 100b defines a groove 111b. These grooves 111a and 111b will be used to latch the couplings 100a and 100b together, as described further below.

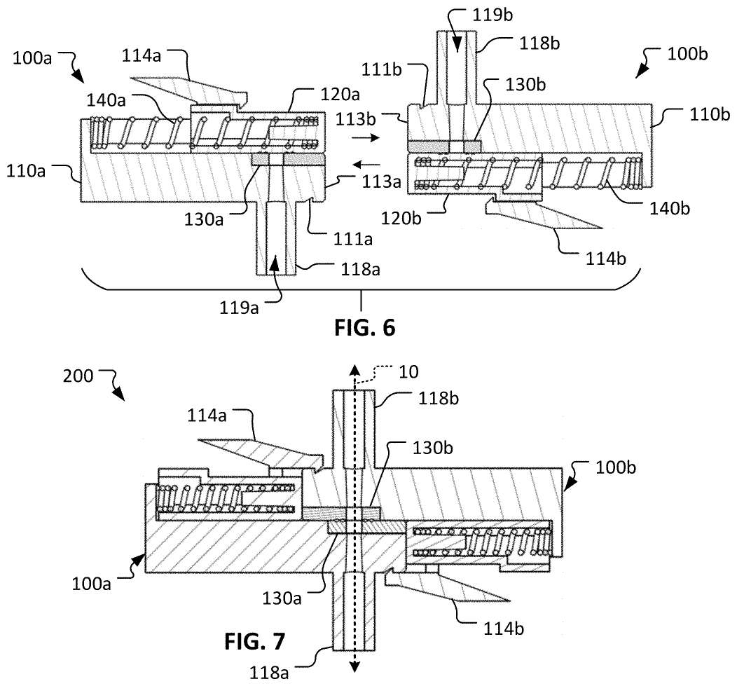

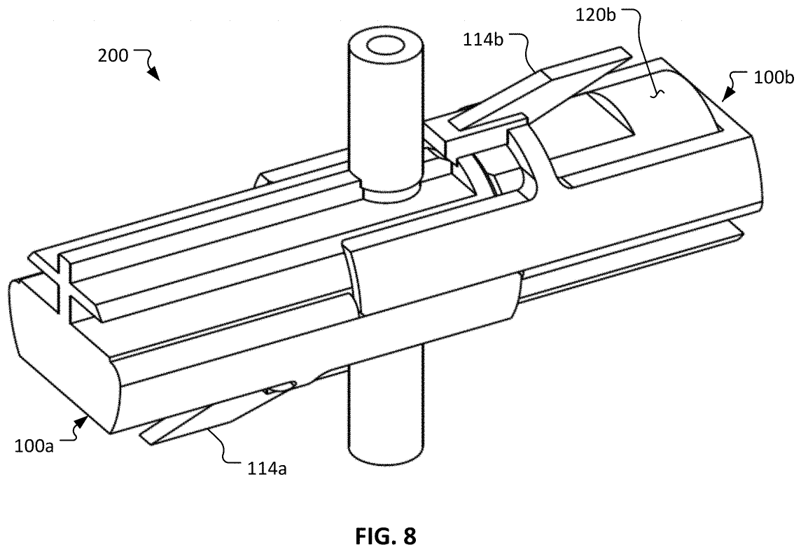

Now referring also to FIG. 7, in this cross-sectional view the first coupling 100a and the second coupling 100b have now been coupled together in an operative arrangement to form a fluid coupling system 200. The fluid coupling system 200 defines an open fluid flow path 10 through the couplings 100a-b (through the lumens 119a-b and the through-holes 132a-b of the seals 130a-b). The open fluid flow path 10 extends between the free ends of the terminations 118a-b.

By comparing the uncoupled arrangement of FIG. 6 to the coupling arrangement of FIG. 7, a number of physical structures and interactions related to the coupling process can be envisioned. For example, when first coupling 100a and the second coupling 100b are pushed toward each other, the front face 113a of the first coupling 100a presses against the shuttle 120b of the second coupling 100b. Likewise, the front face 113b of the second coupling 100b presses against the shuttle 120a of the first coupling 100a. That pressing moves the shuttles 120a-b, thereby compressing the springs 140a and 140b. The front face 113a of the first coupling 100a enters into the internal space 112b (FIG. 13) defined by the second coupling 100b as it pushes the shuttle 120b. Likewise, the front face 113b of the second coupling 100b enters into the internal space 112a (FIG. 13) defined by the first coupling 100a as it pushes the shuttle 120a. As these motions take place, the shuttles 120a-b slide over the seal members 130a-b until the shuttles 120a-b are no longer in contact with the seal members 130a-b. Instead, the seal members 130a and 130b slide across each other and become abutted against each other as shown in FIG. 7. The circular ribs 134a and 134b (FIG. 12) and the through-hole 132 of the seal member 130a become aligned with the circular ribs 134a and 134b and the through-hole 132 of the seal member 130b. This alignment forms a liquid-tight seal.

As the first coupling 100a and the second coupling 100b are pushed toward each other to operatively couple the first coupling 100a and the second coupling 100b, the latch members 114a-b do not need to be manually manipulated. Then, when the first coupling 100a and the second coupling 100b are fully coupled together, the at least one projection 115a-b (FIG. 13) of the latch members 114a-b will automatically snap into the grooves 111a-b. That is, the at least one projection 115a of the latch member 114a will naturally snap into engagement with the groove 111b of the housing 110b. Similarly, the at least one projection 115b of the latch member 114b will naturally snap into engagement with the groove 111a of the housing 110a. This arrangement is depicted in FIG. 7. When the latch members 114a-b have engaged within the grooves 111a-b, the first coupling 100a and the second coupling 100b will be retained together in the coupled, operative arrangement to form the fluid coupling system 200 with the open fluid flow path 10.

To uncouple the first coupling 100a and the second coupling 100b a user will simultaneously depress the depressible arms 117a (FIG. 13) and 117b to unlatch the couplings 100a-b from each other. The springs 140a-b will tend to naturally push the couplings 100a-b apart from each other.

As the first coupling 100a and the second coupling 100b are coupled together (i.e., transitioned from the arrangement of FIG. 6 to the arrangement of FIG. 7), the shuttles 120a-b wipe across the seal members 130a-b, and the seal members 130a-b wipe across each other. Similarly (except in reverse), as the first coupling 100a and the second coupling 100b are uncoupled from each other (i.e., transitioned from the arrangement of FIG. 7 to the arrangement of FIG. 6), the seal members 130a-b wipe across each other, and the shuttles 120a-b wipe across the seal members 130a-b.

These wiping actions are advantageous for multiple reasons. First, when the first coupling 100a and the second coupling 100b are coupled together no surfaces that are exposed to the ambient ever contact areas that are wetted by a fluid contained in the lumens 119a-b. Accordingly, a sterile connection can be made. The same holds true when the first coupling 100a and the second coupling 100b are uncoupled from each other, i.e., no surfaces that are exposed to the ambient ever contact areas that are wetted by a fluid contained in the lumens 119a-b. Accordingly, a sterile disconnection can be made in some cases, and/or virtually no inclusion (captured ambient air) will be induced into the fluid flow paths of the couplings 100a-b. Moreover, no wetted surfaces are ever exposed to ambient. For these reasons, the first coupling 100a and the second coupling 100b can, under some circumstances, be used in a sterile manner over the course of multiple cycles of connection and disconnection. In some cases, sterile wiping of the portions of the fluid couplings 100a-b may help facilitate use of the fluid couplings 100a-b in a sterile manner over the course of multiple cycles of connection and disconnection.

Moreover, the wiping actions that take place during coupling and uncoupling of the first coupling 100a and the second coupling 100b advantageously prevent fluid spillage when uncoupling and air inclusion when coupling.

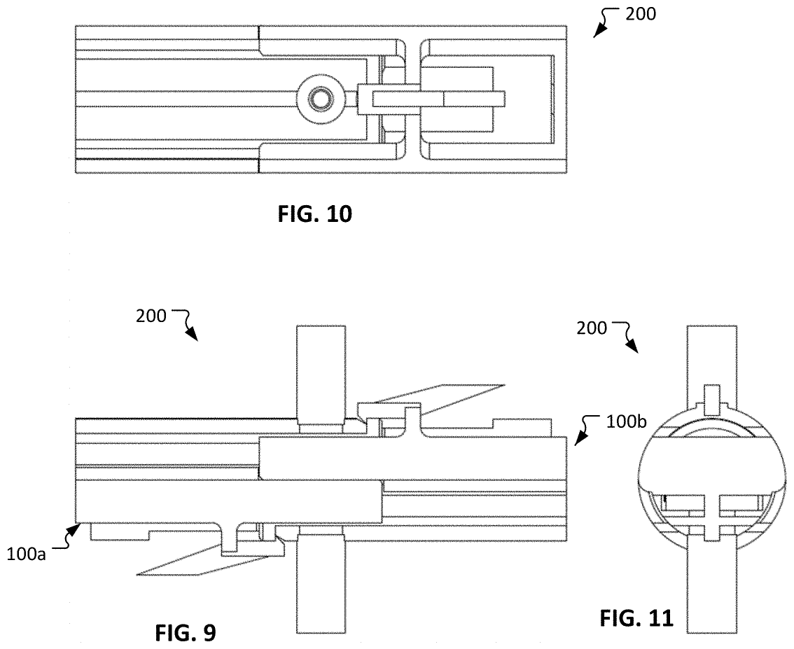

FIGS. 8-11 show additional views of the fluid coupling system 200.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A fluid coupling device comprising:
a housing defining an internal space and a lumen configured for fluid flow, the lumen defining a central axis;
a seal member coupled to the housing and including an upper surface exposed to the internal space, the seal member defining a through-hole in alignment with the lumen; and
a shuttle disposed within the internal space and slidable relative to the housing along a path that intersects the central axis, the shuttle being slidable between: (i) a first position in which the shuttle is abutted against the upper surface of the seal member to cover the through-hole and prevent fluid flow through the lumen, and (ii) a second position in which the through-hole is uncovered by the shuttle.

2. The fluid coupling device of claim 1, further comprising a spring disposed between the housing and the shuttle, wherein the spring biases the shuttle toward the first position.

3. The fluid coupling device of claim 1, further comprising a latch member attached to the housing, wherein the latch member detains the shuttle member in the first position as the spring pushes the shuttle member into contact against the latch member.

4. The fluid coupling device of claim 3, wherein the latch member is manually pivotable relative to the housing.

5. The fluid coupling device of claim 1, wherein the path that the shuttle slides along relative to the housing is orthogonal to the central axis.

6. The fluid coupling device of claim 1, wherein the seal member includes a circular rib that is concentric with the through-hole.

7. The fluid coupling device of claim 1, wherein the seal member includes two circular ribs that are each concentric with the through-hole.

8. The fluid coupling device of claim 1, wherein while the shuttle is in the second position, the lumen is open to the internal space.

9. The fluid coupling device of claim 1, wherein a cross-sectional shape of the internal space taken parallel to the central axis is semicircular.

10. The fluid coupling device of claim 1, wherein a cross-sectional shape of the shuttle taken parallel to the central axis is semicircular.

11. The fluid coupling device of claim 10, wherein the housing has a semicircular front face with a size that matches the cross-sectional shape of the shuttle.

12. A fluid coupling system, comprising:
a first fluid coupling device; and
a second fluid coupling device that is identical to the first fluid coupling device, wherein the first fluid coupling device comprises:
a first housing defining a first internal space and a first lumen configured for fluid flow, the first lumen defining a first central axis;
a first seal member coupled to the first housing and including a first upper surface exposed to the first internal space, the first seal member defining a first through-hole in alignment with the first lumen; and
a first shuttle disposed within the first internal space and slidable relative to the first housing along a first path that intersects the first central axis, the first shuttle being slidable between: (i) a first position in which the first shuttle is abutted against the first upper surface of the first seal member to cover the first through-hole and prevent fluid flow through the first lumen, and (ii) a second position in which the through-hole is uncovered by the shuttle.

13. The fluid coupling system of claim 12, wherein the first and second fluid couplings are slidable relative to each other into a coupled arrangement that defines an unobstructed open fluid flow path therethrough.

14. The fluid coupling system of claim 12, wherein the second fluid coupling device comprises:
a second housing defining a second internal space and a second lumen configured for fluid flow, the second lumen defining a second central axis;
a second seal member coupled to the second housing and including a second upper surface exposed to the second internal space, the second seal member defining a second through-hole in alignment with the second lumen; and
a second shuttle disposed within the second internal space and slidable relative to the second housing along a second path that intersects the second central axis, the second shuttle being slidable between: (i) a first position in which the second shuttle is abutted against the second upper surface of the second seal member to cover the second through-hole and prevent fluid flow through the second lumen, and (ii) a second position in which the second through-hole is uncovered by the second shuttle.

15. The fluid coupling system of claim 14, wherein the first housing and the second housing each have a front face, and wherein a shape of the front face matches a shape of a cross-sectional shape of the first shuttle and a cross-sectional shape of the second shuttle.

16. The fluid coupling system of claim 15, wherein the first and second fluid coupling devices are coupleable by: (i) aligning the first and second fluid coupling devices such that the front face of the first housing is in contact with the second shuttle and the front face of the second housing is in contact with the first shuttle, and (ii) pushing the first and second fluid coupling devices toward each other to slide the first and second shuttles to their second positions.

17. The fluid coupling system of claim 16, wherein when the first and second shuttles are in their second positions the first and second through-holes are in alignment with each other.

18. The fluid coupling system of claim 16, wherein the first fluid coupling device includes a first latch member, wherein the second fluid coupling device includes a second latch member, and wherein when the first and second shuttles are in their second positions: (i) the first latch member is releasably engaged with the second housing and (ii) the second latch member is releasably engaged with the first housing.

19. The fluid coupling system of claim 18, wherein the first latch member is manually pivotable relative to the first housing, wherein the second latch member is manually pivotable relative to the second housing.

20. The fluid coupling system of claim 18, wherein, when the first and second fluid coupling devices are uncoupled from each other, a first spring of the first fluid coupling device forces the first shuttle into engagement against the first latch member and the first latch member detains the first shuttle in the first position against the forces of the first spring.
